(19) United States
(12) Patent Application Publication
(10) Pub. No.: US 2025/0376440 A1
(43) Pub. Date: Dec. 11, 2025

(54) PROCESS AND PLANT FOR PRODUCING UREA

(71) Applicant: JOINT STOCK COMPANY RESEARCH AND DESIGN INSTITUTE OF UREA (JSC NIIK), Dzerzhinsk (RU)

(72) Inventors: Ivan Vladimirovich GUSEV, Dzerzhinsk (RU); Yury Andreevich SERGEEV, Dzerzhinsk (RU); Aleksei Vladimirovich SOLDATOV, Dzerzhinsk (RU); Aleksandr Alekseevich PROKOPYEV, Dzerzhinsk (RU); Sergei Vyacheslavovich SUVORKIN, Dzerzhinsk (RU); Igor Veniaminovich ESIN, Dzerzhinsk (RU)

(73) Assignee: JOINT STOCK COMPANY RESEARCH AND DESIGN INSTITUTE OF UREA (JSC NIIK), Dzerzhinsk (RU)

(21) Appl. No.: 19/301,032

(22) Filed: Aug. 15, 2025

Related U.S. Application Data
(63) Continuation of application No. PCT/RU2024/050040, filed on Feb. 15, 2024.

(30) Foreign Application Priority Data
Feb. 16, 2023 (RU) 2023103809

(51) Int. Cl. C07C 273/04; B01D 3/00; B01D 3/14; B01D 5/00; B01J 4/00; B01J 19/00; B01J 19/24; B01J 19/32; C07C 273/16; F28F 1/00

(52) U.S. Cl. CPC C07C 273/04; B01D 3/007; B01D 3/148; B01D 5/006; B01J 4/001; B01J 19/0013; B01J 19/24; B01J 19/32; C07C 273/16; F28F 1/00; B01J 2204/002; B01J 2219/00103; B01J 2219/0011; B01J 2219/00162

(57) ABSTRACT

A process for producing urea includes reacting ammonia and carbon dioxide in a synthesis zone at elevated temperatures and pressure to form urea synthesis solution, successive distillation of urea synthesis solution at high, medium and at low-pressure step, condensation-absorption of distillation gases to form ammonium carbamate aqueous solutions (CAS), successive recirculation of CAS, and evaporation of urea aqueous solution. At the high-pressure step, urea synthesis solution is distilled in two zones: in the first zone urea synthesis solution is subjected to pressure reduction of at least 0.01-0.4 MPa lower than the synthesis pressure, and to adiabatic separation, the second zone includes distillation through stripping with a carbon dioxide stream. The result is an increase in the specific capacity of the urea synthesis reactor, and a reduction of the gas and heat load on the stripper-distiller.

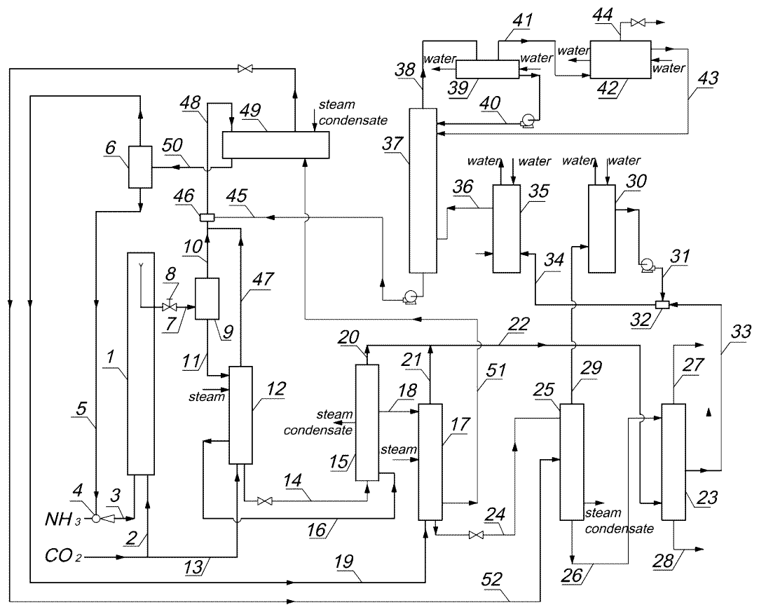

### CROSS-REFERENCE TO RELATED APPLICATIONS

This application is a continuation of International Application No. PCT/RU2024/050040, filed on Feb. 15, 2024, which claims priority to Russian Patent Application No. 2023103809, filed on Feb. 16, 2023. The disclosures of the above-mentioned applications are hereby incorporated by reference in their entireties.

### TECHNICAL FIELD

The application relates to processes and plants for producing urea from ammonia and carbon dioxide.

### BACKGROUND

The process for producing urea is known comprising reaction of ammonia and carbon dioxide in a synthesis zone at elevated temperatures and pressures with formation of urea synthesis solution containing urea, water, ammonium carbamate, ammonia and carbon dioxide, decomposition of ammonium carbamate in urea synthesis solution with heat supply at several pressure steps with formation of urea aqueous solution and gas streams, condensation-absorption of gas streams with water absorbents and formation of ammonium carbamate aqueous solution recirculated into the synthesis zone, evaporation of urea aqueous solution and formation of solid urea (V. Kucheriavy, V. Lebedev. Synthesis and Application of Urea, Leningrad: Khimiya, 1970, p. 191-199).

Urea producing process is known comprising reaction of ammonia and carbon dioxide in the synthesis zone at elevated temperature and pressure (14 MPa) with formation of urea synthesis solution containing urea, water, ammonium carbamate, ammonia and carbon dioxide, distillation of urea synthesis solution with heat supplied sequentially at high-pressure step at the pressure equal to the synthesis pressure and at low-pressure step at 0.4 MPa, with formation of aqueous urea solution and gas streams, condensation-absorption of gas streams with water absorbents and with formation of ammonium carbamate aqueous solution recirculated into the synthesis zone, evaporation of urea aqueous solution and obtaining of solid urea, at the same time urea synthesis solution is removed from the lower part of the synthesis zone, and unreacted gas phase is removed from the upper part of the synthesis zone and sent to gas condensation-absorption stage of high-pressure distillation step (Jozef H. Meessen. Urea. Ullmann's Encyclopedia of industrial chemistry, vol. 37, 2011, p. 670-672).

In this process, urea synthesis solution formed in the synthesis reactor located in the synthesis zone is removed from the lower part of the synthesis reactor through the overflow pipe located inside the synthesis reactor, and in the upper part of the synthesis reactor above the overflow pipe neck a level is maintained that ensures supply of urea synthesis solution through the overflow pipe and removal of gas phase from the reactor upper part by implementing in such a way the process of urea synthesis solution separation directly in the synthesis reactor that requires a significant space volume filled with gas phase. Thus, a significant disadvantage of this process is incomplete use of the reaction volume of the synthesis reactor, and, consequently, low yield of the finished product from a unit volume of the synthesis reactor which amounts to 350-470 kg/(m3h) according to this process.

Urea producing process is known comprising reaction of ammonia and carbon dioxide in the synthesis zone at elevated temperature and pressure (20 MPa) with formation of urea synthesis solution containing urea, water, ammonium carbamate, ammonia and carbon dioxide, distillation of urea synthesis solution with heat supplied from an external source sequentially at high-pressure step at 8-12 MPa, at medium-pressure step at 1.5-2.5 MPa and low-pressure step at 0.2-0.5 MPa, with formation of urea aqueous solution and distillation gases, condensation-absorption of distillation gases with water absorbents when cooling and formation of ammonium carbamate aqueous solution, recirculation of ammonium carbamate aqueous solution from low-pressure distillation gases condensation-absorption stage to medium-pressure distillation gases condensation-absorption stage and from medium-pressure distillation gases condensation-absorption stage to high-pressure distillation gases condensation-absorption stage, and from high-pressure distillation gases condensation-absorption stage to the synthesis zone, evaporation of urea aqueous solution and its subsequent treatment by known processes, in this case distillation of urea synthesis solution at high-pressure step is carried out sequentially in two zones, in the first zone adiabatic separation is carried out, and in the second zone distillation is carried out with heat supply from an external source in carbon dioxide stream (RU 2499791, C07C 273/04, 2013).

The disadvantage of this process is the large pressure difference between the synthesis zone and the urea synthesis solution distillation zone at heat supply in carbon dioxide stream at high-pressure step which is carried out in a film heat-exchanger which is a stripper-distiller since when pressure decreases, efficiency of distillation process by stripping process decreases.

The closest to the proposed process in technical essence is the known process for producing urea by interaction of ammonia and carbon dioxide in the synthesis zone at elevated temperature and pressure to form a urea synthesis solution containing urea, water, ammonium carbamate, ammonia and carbon dioxide, distillation of urea synthesis solution with heat supply from an external source sequentially at high-pressure step equal to the synthesis pressure, at 15.0 MPa, at medium-pressure step at 1.8 MPa and low-pressure step at 0.45 MPa to form a urea aqueous solution and distillation gases, condensation-absorption of distillation gases with water absorbents when cooling to form ammonium carbamate aqueous solutions, recirculation of ammonium carbamate aqueous solution from the low-pressure distillation gases condensation-absorption stage to medium-pressure distillation gases condensation-absorption stage and from medium-pressure distillation gases condensation-absorption stage to high-pressure distillation gases condensation-absorption stage, and from high-pressure distillation gases condensation-absorption stage to the synthesis zone, evaporation of urea aqueous solution in several stages, obtaining solid urea (U.S. Pat. No. 4,314,077, C07C 273/04, 1982).

The disadvantages of this process are incomplete use of the reaction volume of the synthesis reactor located in the synthesis zone when yield of the finished product per unit volume of the synthesis reactor is 400-500 kg/(m3h) and an important load of gas phase on the film heat-exchanger which is a stripper-distiller located in the distillation zone of urea synthesis solution with heat supply at high-pressure steps. Another significant disadvantage is necessity to maintain a relatively high temperature of 200-210° C. in this film heat-exchanger (using heating steam with temperature of 215-225° C.) at which insufficient corrosion resistance of stainless steels is observed that forces to use expensive titanium and zirconium in manufacturing of heat-exchange tubes of the film heat-exchanger that results in a significant rise of equipment price and an increase in the cost for its maintenance.

There is known a plant for producing urea which includes a urea synthesis reactor, units for distillation at several pressure steps of urea synthesis solution formed during synthesis, units for evaporation of urea aqueous solution formed at the last step of distillation, units for condensation-absorption of distillation gases, means for feeding ammonia and carbon dioxide to the urea synthesis reactor, urea synthesis solution from the synthesis reactor to distillation units, urea aqueous solution from the unit of last step distillation to evaporation units, distillation gases from the distillation units to condensation-absorption units, ammonium carbamate aqueous solution from the lower pressure distillation unit to the higher pressure distillation unit and from the higher pressure distillation unit to the synthesis reactor (V. Kucheriavy, V. Lebedev. Synthesis and Application of Urea, Leningrad: Khimiya, 1970, p. 191-199).

There is known a plant for producing urea which includes a urea synthesis reactor, a unit with heat supply from an external source for distillation of urea synthesis solution formed in the synthesis reactor at high-pressure step, a unit with heat supply for distillation of urea synthesis solution at low-pressure step, units for condensation-absorption when cooling of distillation gases formed at high and low-pressure steps, units for evaporation when heating of urea aqueous solution formed at distillation at low-pressure step, means for feeding ammonia and carbon dioxide into the urea synthesis reactor, urea synthesis solution from the synthesis reactor to the distillation unit at high-pressure step, urea synthesis solution from the distillation unit at high-pressure step to the distillation unit at low-pressure step, urea aqueous solution from the distillation unit at low-pressure step to units for evaporation, distillation gases from the distillation unit at high-pressure step to the unit for condensation-absorption of distillation gases formed at high-pressure stage, distillation gases from the distillation unit at low-pressure step to the unit for condensation-absorption of distillation gases at low-pressure step, ammonium carbamate aqueous solution from the unit for condensation-absorption of distillation gases at low-pressure step to the unit for condensation-absorption of distillation gases at high-pressure step and from the unit for condensation-absorption of distillation gases at high-pressure step to the synthesis reactor, at the same time in the lower part of the synthesis reactor there are means for feeding urea synthesis solution from the synthesis reactor to the distillation unit at high-pressure step and in the upper part of the synthesis reactor there are means for feeding unreacted gas phase to the unit for condensation-absorption of distillation gases at high-pressure step (Jozef H. Meessen. Urea. Ullmann's Encyclopedia of industrial chemistry, vol. 37, 2011, p. 670-672).

There is known a plant for producing urea which includes a urea synthesis reactor, a unit with heat supply from an external source for distillation of urea synthesis solution formed in the synthesis reactor at high-pressure step below synthesis pressure, a unit with heat supply for distillation of urea synthesis solution at medium-pressure step, a unit with heat supply for distillation of urea synthesis solution at low-pressure step, units for evaporation when heating urea aqueous solution formed at distillation at low-pressure step, units for condensation-absorption when cooling distillation gases formed at high, medium and low-pressure steps, means for feeding ammonia and carbon dioxide to the urea synthesis reactor, urea synthesis solution from the synthesis reactor to the distillation unit at high-pressure step, urea synthesis solution from the distillation unit at high-pressure step to the distillation unit at medium-pressure step, urea synthesis solution from the distillation unit at medium-pressure step to the distillation unit at low-pressure stage, urea aqueous solution from the distillation unit at low-pressure stage to the evaporation units, distillation gases from the distillation unit at high-pressure step to the unit for condensation-absorption of distillation gases at high-pressure step, distillation gases from the distillation unit at medium-pressure step to the unit for condensation-absorption of distillation gases at medium-pressure step, distillation gases from the distillation unit at low-pressure step to the unit for condensation-absorption of distillation gases at low-pressure step, ammonium carbamate aqueous solution from the unit for condensation-absorption of distillation gases at low-pressure step to the unit for condensation-absorption of distillation gases at medium-pressure step, ammonium carbamate aqueous solution from the unit for condensation-absorption of distillation gases at medium-pressure step to the unit for condensation-absorption of distillation gases at high-pressure step and from the unit for condensation-absorption of distillation gases at high-pressure step into the synthesis reactor, at the same time the distillation unit at high-pressure step consists of a high-pressure separator and a film heat-exchanger and the plant contains means for feeding urea synthesis solution from the high-pressure separator into the film heat-exchanger, means for feeding carbon dioxide into the film heat-exchanger (RU 2499791, C07C 273/04, 2013).

The closest to the proposed one is the plant for producing urea which includes a urea synthesis reactor, a unit with heat supply from an external source for distillation of urea synthesis solution formed in the synthesis reactor at high-pressure step, a unit with heat supply for distillation of urea synthesis solution at medium-pressure step, a unit with heat supply for distillation of urea synthesis solution at low-pressure step, a unit for pre-evaporation of urea aqueous solution formed at distillation at low-pressure step, a unit for following evaporation of urea aqueous solution, units for condensation-absorption when cooling distillation gases formed at high, medium and low-pressure steps, means for feeding ammonia and carbon dioxide to the urea synthesis reactor, urea synthesis solution from the synthesis reactor to the unit for distillation at high-pressure step, urea synthesis solution from the unit for distillation at high-pressure step to the unit for distillation at medium-pressure step, urea synthesis solution from medium-pressure distillation unit to the low-pressure distillation unit, urea aqueous solution from the low-pressure distillation unit to the pre-evaporation unit and from the pre-evaporation unit to the post-evaporation unit, distillation gases from the high-pressure distillation unit to the unit for condensation-absorption of distillation gases at high-pressure step, distillation gases from the medium-pressure distillation unit to the unit for condensation-absorption of distillation gases at medium-pressure step, distillation gases from the low-pressure distillation unit to the unit for condensation-absorption of distillation gases at low-pressure step, ammonium carbamate aqueous solution from the unit for condensation-absorption of distillation gases at low-pressure step to the unit for condensation-absorption of distillation gases at medium-pressure step, ammonium carbamate aqueous solution from the unit for condensation-absorption of distillation gases at medium-pressure step into the unit for condensation-absorption of distillation gases at high-pressure step, and from the unit for condensation-absorption of distillation gases at high-pressure step into the synthesis reactor (U.S. Pat. No. 4,314,077, C07C 273/04, 1982).

### SUMMARY

The problem solved by the proposed application consists in improvement of the known process and plant for urea production as well as in increase of their energy efficiency and cost effectiveness.

The technical result obtained at implementation of the application consists in increase of the specific production capacity of urea synthesis reactor, reduction of gas and heat load on the film heat-exchanger of the distillation unit at high-pressure step, which is a stripper-distiller, as well as reduction of stripper-distiller cost.

In order to achieve this result, there is provided a process for producing urea by reacting ammonia and carbon dioxide in a synthesis zone at elevated temperature and pressure to form a urea synthesis solution containing urea, water, ammonium carbamate, ammonia and carbon dioxide, followed by distillation of the urea synthesis solution with heat supply from an external source successively at a high-pressure step at 14.0-16.5 MPa, at a medium-pressure step at 1.5-2.5 MPa and at a low-pressure step at 0.2-0.5 MPa to form a urea aqueous solution and distillation gases, condensation-absorption of the distillation gases with water absorbents when cooling to form ammonium carbamate aqueous solutions, ammonium carbamate aqueous solution recirculation from a low-pressure distillation gases condensation-absorption stage to a medium-pressure distillation gases condensation-absorption stage, from the medium-pressure distillation gases condensation-absorption stage to a high-pressure distillation gases condensation-absorption stage, and from the high-pressure distillation gases condensation-absorption stage to the synthesis zone, evaporation of the urea aqueous solution in several stages, wherein distillation of the urea synthesis solution at the high-pressure step is carried out in two consecutive zones, in the first zone adiabatic pressure reduction of the urea synthesis solution is carried out to a pressure of at least 0.01-0.4 MPa lower than the synthesis pressure and following adiabatic separation is carried out, and in the second zone distillation is carried out with heat supply from an external source by means of stripping with a carbon dioxide stream.

In order to achieve this result, there is also provided a plant for producing urea, comprising a urea synthesis reactor, a unit with heat supply from an external source for distillation of a urea synthesis solution formed in the synthesis reactor at a high-pressure step, a unit with heat supply for distillation of the urea synthesis solution at a medium-pressure step, a unit with heat supply for distillation of the urea synthesis solution at a low-pressure step, a recuperative heat-exchanger for pre-evaporation of a urea aqueous solution formed at distillation at the low-pressure step, a unit for following evaporation of the urea aqueous solution, units for condensation-absorption when cooling of distillation gases formed at the high-pressure, medium-pressure and low-pressure steps, means for feeding ammonia and carbon dioxide into the urea synthesis reactor, means for feeding the urea synthesis solution from the synthesis reactor to the unit for distillation at the high-pressure step, means for feeding the urea synthesis solution from the unit for distillation at the high-pressure step to the unit for distillation at the medium-pressure step, means for feeding the urea synthesis solution from the unit for distillation at the medium-pressure step to the unit for distillation at the low-pressure step, means for feeding the urea aqueous solution from the unit for distillation at the low-pressure step to the recuperative heat-exchanger and from the recuperative heat-exchanger to the unit for following evaporation, means for feeding the distillation gases from the unit for distillation at the high-pressure step to the unit for condensation-absorption of distillation gases at the high-pressure step, means for feeding distillation gases from the unit for distillation at the medium-pressure step to the unit for condensation-absorption of distillation gases at the medium-pressure step, means for feeding distillation gases from the unit for distillation at the low-pressure step to the unit for condensation-absorption of distillation gases at the low-pressure step, means for feeding an ammonium carbamate aqueous solution from the unit for condensation-absorption of distillation gases at the low-pressure step to the unit for condensation-absorption of distillation gases at the medium-pressure step, means for feeding the ammonium carbamate aqueous solution from the unit for condensation-absorption of distillation gases at the medium-pressure step to the unit for condensation-absorption of distillation gases at the high-pressure step, and from the unit for condensation-absorption of distillation gases at the high-pressure step to the synthesis reactor, wherein the unit for distillation at the high-pressure step comprises a high-pressure separator and a film heat-exchanger, and the plant additionally comprises means for feeding the urea synthesis solution from the high-pressure separator to the film heat-exchanger and means for feeding fresh carbon dioxide to the film heat-exchanger, and means for feeding the urea synthesis solution from the synthesis reactor to the unit for distillation at the high-pressure step are fitted with a device for pressure reduction by 0.01-0.4 MPa.

Within the application it is possible to implement its different modification, which are special cases of its implementation and allow to increase the degree of heat recovery of the production cycle.

In one modification of the process, the medium-pressure distillation gases are fed to the medium-pressure distillation gases condensation-absorption stage after their heat exchange through a wall with the urea aqueous solution at a pre-evaporation stage. In this case, the plant comprises the means for feeding distillation gases from the unit for distillation at the medium-pressure step to the unit for condensation-absorption of distillation gases at the medium-pressure step comprise means for feeding the distillation gases from the unit for distillation at the medium-pressure step to the recuperative heat-exchanger and from the recuperative heat-exchanger to the unit for condensation-absorption of distillation gases at the medium-pressure step.

In the other modification of the process, the condensation-absorption of the high-pressure distillation gases is carried out in two consecutive zones, in the first zone condensation is carried out, and in the second zone adiabatic separation is carried out. In this case, the plant comprises the unit for condensation-absorption of distillation gases at the high-pressure step comprises a high-pressure condenser and a high-pressure separator, and the plant additionally comprises means for feeding the ammonium carbamate aqueous solution from the high-pressure condenser to the high-pressure separator.

In the third modification of the process, the distillation of the urea synthesis solution at the low-pressure step is carried out by heat exchange through a wall with saturated water steam formed in the condensation zone at the high-pressure distillation gases condensation-absorption stage. In this case, the plant additionally comprises means for feeding saturated water steam from the high-pressure condenser of the unit for condensation-absorption of distillation gases at the high-pressure step to the heating zone of the unit for distillation at the low-pressure step.

In the fourth modification of the process, the distillation of the urea synthesis solution at the medium-pressure step is carried out in two consecutive zones, in the first zone distillation is carried out by heat exchange through a wall with a steam condensate formed in the distillation zone with heat supply at the high-pressure step, and in the second zone distillation is carried out with heat supply in a stream of inert gases formed in the separation zone of the ammonium carbamate aqueous solution at the high-pressure distillation gases condensation-absorption stage. In this case, the plant comprises the unit for at the medium-pressure step comprises a medium-pressure heat-exchanger and a medium-pressure distiller, and the plant further comprises means for feeding steam condensate from the film heat-exchanger of the unit for distillation at the high-pressure step to the heating zone of the medium-pressure heat-exchanger and means for feeding inert gases from the high-pressure separator of the unit for condensation-absorption of distillation gases at the high-pressure step to the heating zone of the medium-pressure distiller.

In the fifth modification of the process, in the first zone of the urea synthesis solution distillation at the high-pressure step adiabatic pressure reduction of the urea synthesis solution is carried out to a pressure of 0.1-0.2 MPa lower than the synthesis pressure. In this case the plant includes means for feeding the urea synthesis solution from the synthesis reactor to the unit for distillation at the high-pressure step are fitted with a device for pressure reduction by 0.1-0.2 MPa.

If adiabatic separation of urea synthesis solution which is supplied from the synthesis reactor is arranged outside the synthesis reactor, then this allows to use the maximum possible volume of the synthesis reactor for the reaction mass due to reduction of free volume in the top part of the synthesis reactor filled with gas phase. At the same time, the total volume of the synthesis reactor remains unchanged. Increase of the volume of the synthesis reactor filled with reaction mass allows to place a larger quantity of mass-exchanging trays inside the synthesis reactor as well as to increase the residence time of the reaction mass in the reactor, what results in urea synthesis reactor capacity increase per unit of the reactor volume (specific capacity of synthesis reactor) to 600 kg/(m3h), in comparison with 370-500 kg/(m3h) for similar plants.

Urea synthesis solution at the outlet from the synthesis reactor is a gas-liquid mixture, which is in a state of phase equilibrium. Insignificant (by 0.01-0.4 MPa) adiabatic pressure reduction of urea synthesis solution leaving the synthesis reactor shifts the phase equilibrium of the gas-liquid mixture and then allows to carry out adiabatic separation of urea synthesis solution. As a result, it is possible to separate effectively about 10% of the gas phase mass from urea synthesis solution, what allows to reduce the load on the film heat-exchanger of the unit for distillation at high-pressure step, that is a stripper-distiller, and to increase the efficiency of its operation.

Supply of fresh carbon dioxide to the film heat-exchanger of the unit for distillation at high-pressure step allows, due to increase of the thermal potential of high-pressure distillation gases, to carry out the decomposition process of unreacted ammonium carbamate at lower temperature of 185-200° C. (using heating steam with temperature of 212-215° C.), at which it is possible to use stainless steels for manufacturing of heat-exchange tubes for the film heat-exchanger of the unit for distillation at high-pressure step instead of expensive titanium and zirconium, what reduces significantly the cost of the film heat-exchanger.

### BRIEF DESCRIPTION OF THE DRAWINGS

The accompanying FIGURE shows the process flow diagram of the plant for producing urea in accordance with the claimed application.

In according with the accompanying FIGURE, the plant comprises:
1—urea synthesis reactor;
2—stream of gaseous carbon dioxide;
3—stream of liquid ammonia;
4—high-pressure ejector;
5—stream of ammonium carbamate aqueous solution from high-pressure separator 6;
6—high-pressure separator;
7—stream of urea synthesis solution;
8—pressure reduction valve;
9—high-pressure separator;
10—gas stream from high-pressure separator 9;
11—stream of urea synthesis solution from high-pressure separator 9;
12—stripper-distiller;
13—stream of gaseous carbon dioxide;
14—stream of urea synthesis solution;
15—pre-decomposer;
16—stream of steam condensate from stripper-distiller 12;
17—medium-pressure distiller;
18—stream of urea synthesis solution from pre-decomposer 15;
19—stream of inert gases from high-pressure separator 6;
20—stream of distillation gases from pre-decomposer 15;
21—stream of distillation gases from medium-pressure distiller 17;
22—combined gaseous stream of stream 20 and stream 21;
23—recuperative heat-exchanger;
24—stream of urea synthesis solution from medium-pressure distiller 17;
25—low-pressure distiller;
26—stream of urea aqueous solution from low-pressure distiller 25;
27—stream of secondary steam from recuperative heat-exchanger 23;
28—stream of urea solution from recuperative heat-exchanger 23;
29—stream of distillation gases from low-pressure distiller 25;
30—low-pressure condenser;
31—stream of ammonium carbamate aqueous solution from low-pressure condenser 30;
32—mixer;
33—stream of distillation gases from recuperative heat-exchanger 23;
34—mixed gas-liquid stream of stream 31 and stream 33;
35—medium-pressure condenser;
36—gas-liquid stream from medium-pressure condenser 35;
37—wash column;
38—stream of gaseous ammonia from washing column 37;
39—ammonia condenser;
40—stream of liquid ammonia from ammonia condenser 39;
41—stream of uncondensed ammonia and inert gases from ammonia condenser 39;
42—scrubber;
43—stream of ammonia water from scrubber 42;
44—stream of inert gases from scrubber 42;
45—stream of ammonium carbamate aqueous solution from washing column 37;
46—mixer;
47—stream of distillation gases from stripper-distiller 12;
48—mixed gas-liquid stream of stream 10, stream 45 and stream 47;
49—high-pressure condenser;
50—gas-liquid stream from high-pressure condenser 49;
51—stream of steam condensate from medium-pressure distiller 17; and
52—stream of saturated water steam from high-pressure condenser 49.

### DETAILED DESCRIPTION OF THE EMBODIMENTS

Summary of the application is illustrated by the examples given below with reference to the process flow diagram shown in the attached FIGURE, which are ones of the possible options for implementation of the proposed process and plant.

### Example 1

A stream 2 of gaseous carbon dioxide and a stream 3 of liquid ammonia, which comes from the ammonia network through a high-pressure ejector 4 (the molar ratio of NH₃:CO₂ in the reactor 1 is 3.1:1), are fed into the reactor 1 for urea synthesis together with a stream 5 of ammonium carbamate aqueous solution from a high-pressure separator 6. In the reactor 1, at a pressure of 14.0-15.5 MPa and a temperature of 170-190° C., a reaction of urea synthesis occurs with formation of a urea synthesis solution containing urea, water, ammonium carbamate not converted into urea, excess ammonia, and inert gases.

The urea synthesis solution stream 7 is withdrawn from the upper part of the reactor 1, the pressure of stream 7 is reduced by 0.2 MPa by a pressure reduction valve 8, and then stream 7 is directed to a high-pressure separator 9, where the gas and liquid phases of stream 7 are separated at the synthesis temperature. As a result, a gas stream 10 (10% by weight of the mass of the urea synthesis solution stream 7), containing predominantly unreacted ammonia and carbon dioxide, and a liquid stream 11 of urea synthesis solution released from the unreacted gas phase, are separated from the urea synthesis solution.

The urea synthesis solution stream 11 from the high-pressure separator 9, without changing the pressure, enters a stripper-distiller 12, which is a film heat-exchanger, where, when heated by high-pressure steam (super atmospheric pressure is 2.1 MPa, temperature is 215° C.) by means of stripping with the stream of gases released from the urea synthesis solution and a carbon dioxide stream 13, at 190° C., decomposition of the greater part of ammonium carbamate and distillation of excess ammonia occurs. A urea synthesis solution stream 14 with a concentration of 40-45% and a temperature of 195-200° C., discharged from the stripper-distiller 12, is reduced to a pressure of 1.6-1.8 MPa. Then the urea synthesis solution stream 14 is fed to the lower part of a pre-decomposer 15, which is a medium-pressure heat-exchanger, where, at a temperature of 125-135° C., ammonia, carbon dioxide, and water are distilled from the urea synthesis solution.

The separation zone is located in the upper part of the pre-decomposer 15. The lower part of the pre-decomposer 15 is a vertical shell-and-tube heat-exchanger operating in the submerged mode, into the shell side of which steam condensate from the stripper-distiller 12 goes by stream 16. The pre-decomposer 15 provides preliminary heating and distillation of unreacted components from the urea synthesis solution, thereby reducing the gas and heat load on a medium-pressure distiller 17. The urea synthesis solution is sent from the pre-decomposer 15 by stream 18 to the upper part of the medium-pressure distiller 17, where ammonia, carbon dioxide, and water are distilled from the urea synthesis solution at a temperature of 154° C.

The upper part of the medium-pressure distiller 17 combines the mass-exchanging tray zone and the separation zone. The lower part of the medium-pressure distiller 17 is a film heat-exchanger, into which non-condensed inert gases from the high-pressure separator 6 are fed by stream 19 as a stripping agent. The medium-pressure distillation gases are discharged by stream 20 from the pre-decomposer 15 and by stream 21 from the medium-pressure distiller 17, and by combined stream 22 with a temperature of 135-152° C., are directed to the shell side space of a recuperative heat-exchanger 23.

A urea synthesis solution stream 24 is discharged from the medium-pressure distiller 17 with a concentration of 59-63% and a temperature of 155-165° C., is reduced to a pressure of 0.3 MPa, and fed to a low-pressure distiller 25, where ammonia, carbon dioxide, and water are distilled from the urea synthesis solution at a temperature of 130-140° C. The upper part of the low-pressure distiller 25 combines the mass-exchanging tray zone and the separation zone. The lower part of the low-pressure distiller 25 is a film heat-exchanger. A stream 26 of urea aqueous solution with a concentration of 66-72% is fed from the low-pressure distiller 25 and sent to the recuperative heat-exchanger 23, where, at a residual pressure of 40-60 kPa, final distillation of ammonia and carbon dioxide, as well as evaporation of the urea solution, occurs due to use of condensation heat from the medium-pressure distillation gases supplied by stream 22. Secondary steam from the recuperative heat-exchanger 23 is supplied by stream 27 to the vapor condensation section of an evaporation stage (not shown in the FIGURE). Then the urea solution with a concentration of 75% is withdrawn from the recuperative heat-exchanger 23 and is fed by stream 28 for further evaporation and granulation by known processes (not shown in the FIGURE).

The low-pressure distillation gases are discharged by stream 29 from the upper part of the low-pressure distiller 25 and, with a temperature of 122-135° C., are directed to a low-pressure condenser 30, where absorption and condensation of the low-pressure distillation gases occur when cooling with water to form a dilute ammonium carbamate aqueous solution.

The ammonium carbamate aqueous solution formed in the low-pressure condenser 30 is fed by stream 31 to a mixer 32, where it is mixed with a stream 33 of medium-pressure distillation gases leaving the shell side of the recuperative heat-exchanger 23, after which the obtained mixed stream 34 with a temperature of 95-110° C. enters a medium-pressure condenser 35, and then, by stream 36 with a temperature of 90-100° C., enters a wash column 37 for phase separation and washing the gas phase from carbon dioxide. The gaseous ammonia from the washing column 37 is directed by stream 38 for condensation in an ammonia condenser 39, the resulting liquid ammonia is partially recirculated as reflux in the washing column 37 by stream 40, and the rest is fed into the ammonia network. The uncondensed ammonia and inert gases from the ammonia condenser 39 are directed by stream 41 to a scrubber 42, equipped with a heat-exchanger and an absorption column. The ammonia water formed in the scrubber 42 is fed by stream 43 to irrigate the washing column 37. The inert gases from the scrubber 42 are discharged into the atmosphere by stream 44.

An ammonium carbamate aqueous solution with a temperature of 75-105° C. from the bottom part of the washing column 37 is fed by stream 45 to a mixer 46, where the gas stream 10 from the high-pressure separator 9 is also fed together with a gas stream 47 from the stripper-distiller 12. From the mixer 46, a gas-liquid stream 48 enters a high-pressure condenser 49, where condensation of gases occurs with formation of an ammonium carbamate solution. The gas-liquid mixture from the high-pressure condenser 49 enters the high-pressure separator 6 via stream 50. In the shell side of the high-pressure condenser 49, during evaporation of steam condensate entering via stream 51 from the medium-pressure distiller 17, saturated water steam with a super atmospheric pressure of 0.45 MPa is formed due to the heat of ammonium carbamate formation and ammonia dissolution at 180° C., which is fed by stream 52 to the shell side of the low-pressure distiller 25. In the high-pressure separator 6, the gas-liquid mixture is separated with formation of stream 5 of ammonium carbamate aqueous solution recirculated into the reactor 1 and stream 19 of uncondensed gases, which is fed to the lower part of the medium-pressure distiller 17.

### Example 2

The process is carried out similarly to the example 1, with the difference that the pressure reduction valve 8 reduces the pressure of the urea synthesis solution stream 7 by 0.1 MPa, and then stream 7 is directed to the high-pressure separator 9, where the gas stream 10 is released at the synthesis temperature and contains 7% by weight of the mass of the urea synthesis solution stream 7.

### Example 3

The process is carried out similarly to the example 1, with the difference that the pressure reduction valve 8 reduces the pressure of the urea synthesis solution stream 7 by 0.01 MPa, and then stream 7 is directed to the high-pressure separator 9, where the gas stream 10 is released at the synthesis temperature and contains 1.5% by weight of the mass of the urea synthesis solution stream 7.

### Example 4

The process is carried out similarly to the example 1, with the difference that the pressure reduction valve 8 reduces the pressure of the urea synthesis solution stream 7 by 0.4 MPa, and then stream 7 is directed to the high-pressure separator 9, where the gas stream 10 is released at the synthesis temperature and contains 11% by weight of the mass of the urea synthesis solution stream 7.

The application can be used in the industry for producing urea from ammonia and carbon dioxide.

What is claimed is:

1. A process for producing urea, comprising: reacting ammonia and carbon dioxide in a synthesis zone at elevated temperature and pressure to form a urea synthesis solution containing urea, water, ammonium carbamate, ammonia and carbon dioxide, followed by distillation of the urea synthesis solution with heat supply from an external source successively at a high-pressure step at 14.0-16.5 MPa, at a medium-pressure step at 1.5-2.5 MPa and at a low-pressure step at 0.2-0.5 MPa to form a urea aqueous solution and distillation gases, condensation-absorption of the distillation gases with water absorbents when cooling to form ammonium carbamate aqueous solutions, ammonium carbamate aqueous solution recirculation from a low-pressure distillation gases condensation-absorption stage to a medium-pressure distillation gases condensation-absorption stage, from the medium-pressure distillation gases condensation-absorption stage to a high-pressure distillation gases condensation-absorption stage, and from the high-pressure distillation gases condensation-absorption stage to the synthesis zone, evaporation of the urea aqueous solution in several stages,
wherein distillation of the urea synthesis solution at the high-pressure step is carried out in two consecutive zones, in the first zone adiabatic pressure reduction of the urea synthesis solution is carried out to a pressure of at least 0.01-0.4 MPa lower than the synthesis pressure and following adiabatic separation is carried out, and in the second zone distillation is carried out with heat supply from an external source by means of stripping with a carbon dioxide stream.

2. The process according to claim 1, wherein the medium-pressure distillation gases are fed to the medium-pressure distillation gases condensation-absorption stage after their heat exchange through a wall with the urea aqueous solution at a pre-evaporation stage.

3. The process according to claim 1, wherein condensation-absorption of the high-pressure distillation gases is carried out in two consecutive zones, in a first zone condensation is carried out, and in a second zone adiabatic separation is carried out.

4. The process according to claim 3, wherein distillation of the urea synthesis solution at the low-pressure step is carried out by heat exchange through a wall with saturated water steam formed in the condensation zone at the high-pressure distillation gases condensation-absorption stage.

5. The process according to claim 3, wherein distillation of the urea synthesis solution at the medium-pressure step is carried out in two consecutive zones, in the first zone distillation is carried out by heat exchange through a wall with a steam condensate formed in the distillation zone with heat supply at the high-pressure step, and in the second zone distillation is carried out with heat supply in a stream of inert gases formed in the separation zone of the ammonium carbamate aqueous solution at the high-pressure distillation gases condensation-absorption stage.

6. The process according to claim 1, wherein in the first zone of the urea synthesis solution distillation at the high-pressure step adiabatic pressure reduction of the urea synthesis solution is carried out to a pressure of 0.1-0.2 MPa lower than the synthesis pressure.

7. A plant for producing urea, comprising
a urea synthesis reactor,
a unit with heat supply from an external source for distillation of a urea synthesis solution formed in the synthesis reactor at a high-pressure step,
a unit with heat supply for distillation of the urea synthesis solution at a medium-pressure step,
a unit with heat supply for distillation of the urea synthesis solution at a low-pressure step,
a recuperative heat-exchanger for pre-evaporation of a urea aqueous solution formed at distillation at the low-pressure step,
a unit for following evaporation of the urea aqueous solution,
units for condensation-absorption when cooling of distillation gases formed at the high-pressure, medium-pressure and low-pressure steps,
means for feeding ammonia and carbon dioxide into the urea synthesis reactor,
means for feeding the urea synthesis solution from the synthesis reactor to the unit for distillation at the high-pressure step,
means for feeding the urea synthesis solution from the unit for distillation at the high-pressure step to the unit for distillation at the medium-pressure step,
means for feeding the urea synthesis solution from the unit for distillation at the medium-pressure step to the unit for distillation at the low-pressure step,
means for feeding the urea aqueous solution from the unit for distillation at the low-pressure step to the recuperative heat-exchanger and from the recuperative heat-exchanger to the unit for following evaporation,
means for feeding the distillation gases from the unit for distillation at the high-pressure step to the unit for condensation-absorption of distillation gases at the high-pressure step,
means for feeding distillation gases from the unit for distillation at the medium-pressure step to the unit for condensation-absorption of distillation gases at the medium-pressure step,
means for feeding distillation gases from the unit for distillation at the low-pressure step to the unit for condensation-absorption of distillation gases at the low-pressure step,
means for feeding an ammonium carbamate aqueous solution from the unit for condensation-absorption of distillation gases at the low-pressure step to the unit for condensation-absorption of distillation gases at the medium-pressure step,
means for feeding the ammonium carbamate aqueous solution from the unit for condensation-absorption of distillation gases at the medium-pressure step to the unit for condensation-absorption of distillation gases at the high-pressure step, and from the unit for condensation-absorption of distillation gases at the high-pressure step to the synthesis reactor,
wherein the unit for distillation at the high-pressure step comprises a high-pressure separator and a film heat-exchanger, and the plant additionally comprises means for feeding the urea synthesis solution from the high-pressure separator to the film heat-exchanger and means for feeding fresh carbon dioxide to the film heat-exchanger, and means for feeding the urea synthesis solution from the synthesis reactor to the unit for distillation at the high-pressure step are fitted with a device for pressure reduction by 0.01-0.4 MPa.

8. The plant according to claim 7, wherein the means for feeding distillation gases from the unit for distillation at the medium-pressure step to the unit for condensation-absorption of distillation gases at the medium-pressure step comprise means for feeding the distillation gases from the unit for distillation at the medium-pressure step to the recuperative heat-exchanger and from the recuperative heat-exchanger to the unit for condensation-absorption of distillation gases at the medium-pressure step.

9. The plant according to claim 7, wherein the unit for condensation-absorption of distillation gases at the high-pressure step comprises a high-pressure condenser and a high-pressure separator, and the plant additionally comprises means for feeding the ammonium carbamate aqueous solution from the high-pressure condenser to the high-pressure separator.

10. The plant according to claim 9, wherein the plant additionally comprises means for feeding saturated water steam from the high-pressure condenser of the unit for condensation-absorption of distillation gases at the high-pressure step to the heating zone of the unit for distillation at the low-pressure step.

11. The plant according to claim 9, wherein the unit for distillation at the medium-pressure step comprises a medium-pressure heat-exchanger and a medium-pressure distiller, and the plant further comprises means for feeding steam condensate from the film heat-exchanger of the unit for distillation at the high-pressure step to the heating zone of the medium-pressure heat-exchanger and means for feeding inert gases from the high-pressure separator of the unit for condensation-absorption of distillation gases at the high-pressure step to the heating zone of the medium-pressure distiller.

12. The plant according to claim 7, wherein the means for feeding the urea synthesis solution from the synthesis reactor to the unit for distillation at the high-pressure step are fitted with a device for pressure reduction by 0.1-0.2 MPa.
